(19) United States
(12) Patent Application Publication
(10) Pub. No.: US 2021/0369936 A1
(43) Pub. Date: Dec. 2, 2021

(54) POSTPARTUM BLOOD LOSS COLLECTION AND MEASUREMENT DEVICE, AND METHOD OF USE

(71) Applicant: Measure Co., Pittsburgh, PA (US)

(72) Inventor: Rebecca R.S. Clark, Upper Darby, PA (US)

(73) Assignee: Measure Co., Pittsburgh, PA (US)

(21) Appl. No.: 17/337,036

(22) Filed: Jun. 2, 2021

Related U.S. Application Data
(60) Provisional application No. 63/033,595, filed on Jun. 2, 2020.

(51) Int. Cl. A61M 1/00

(52) U.S. Cl. CPC A61M 1/69; A61M 2205/75; A61M 2205/583; A61M 2205/3379

(57) ABSTRACT

Devices and methods for collecting and measuring postpartum blood loss of a patient are disclosed. The device generally includes a tray that may be positioned under a patient and a basin configured to collect blood. The basin can include gradation markings to facilitate accurate, real-time measurements of the amount of blood that the patient has lost to guide decision making of medical personnel.

### CROSS-REFERENCE TO RELATED APPLICATIONS

This application claims the benefit of U.S. Provisional Application No. 63/033,595, which was filed on Jun. 2, 2020. The entire content of the foregoing provisional patent application is incorporated herein by reference.

### TECHNICAL FIELD

The present invention relates generally to devices and methods of collecting and measuring bodily fluids. In particular, the present invention relates to devices and methods that can be used for measuring postpartum blood loss.

### BACKGROUND

Postpartum hemorrhage (“PPH”) is a serious condition defined by excessive bleeding after childbirth. PPH is a major cause of maternal morbidity and mortality worldwide, affecting approximately 1-5% of women who give birth, but responsible for approximately 11-60% of overall maternal mortality. PPH can manifest anywhere from within one day of giving birth and up to 12 weeks after childbirth.

Visual estimation is traditionally the predominant method of measuring postpartum blood loss. However, because such visual estimation is neither reliable nor accurate, the patient may be under- or over-treated, resulting in potential for significant blood loss to be under-diagnosed. Weighing pads and drapes (also called gravimetry) provide a more reliable method of measuring blood loss. However, because pads and drapes collect all bodily fluids, gravimetry may also be prone to inaccuracy. In addition, gravimetry generally does not easily provide real-time estimates of blood loss because the weighing of collected bodily fluids occurs intermittently, requiring a nurse to be pulled out of circulation. In some instances, a “stat” blood draw from the woman giving birth may be sent to the lab to monitor dropping hemoglobin, but this method can be slow and is therefore incapable of providing real-time estimates for decision making during the operation and/or delivery.

Advancements in technology have been used in an attempt to improve postpartum blood loss. Colorimetry generally involves image capture of a surgical sponge(s) and/or canister(s) with a mobile device, and image recognition algorithms are used to estimate postpartum blood loss from the captured images. Although colorimetry may be a more effective method of measuring blood loss during childbirth (as compared to traditional methods), colorimetry is generally costly and necessitates specialized equipment. Because of the cost and specialized equipment associated with colorimetry, this method is not typically used in obstetrics. Colorimetry is also typically performed at the end of an operation, making it less useful for real-time blood loss measurements during a medical procedure that can guide decision making that can affect the life of the patient.

Thus, there is a need for devices and methods that provide accurate and reliable measurements of postpartum blood loss in real-time without obstructing the clinical workflow around the patient.

### SUMMARY

Embodiments of the present invention provide immediate, real-time assessments of blood loss that are accurate and reliable, without requiring expensive specialized equipment or additional lab tests. In some embodiments, the device can include a measuring cup (e.g., that resembles an upside-down baseball cap) having a tray (e.g., a bill) and a basin (e.g., a cap). The tray can facilitate manipulation and/or positioning of the device relative to the bed and/or the patient, including operable placement of the device during use. The basin can collect blood and provides an accurate and reliable means of measuring the amount of blood that has collected in the device in real-time. In some embodiments, the device can include a plug, nozzle, valve and/or drain at the top or bottom of the cap to allow selective drainage of the basin without moving the entire device. For example, the device can be fluidly connected to a drain line such that when draining of the blood from the basin is desired, a valve or plug can be selectively actuated to allow for drainage through the drain line.

In some embodiments, the device can be fabricated from a thin, medical-grade plastic. In some embodiments, the device can be fabricated from any non-absorbent material suitable for medical use. In some embodiments, the device can be reusable (e.g., capable of being disinfected) or disposable. The dimensions of the device, including the tray and the basin, can be varied depending on the intended use of the device and/or the patient size (e.g., to ensure comfort of the patient). For example, in some embodiments, the device can have a shallower basin and/or a more compact tray. As a further example, in some embodiments, the device can have a deeper basin and/or an extended tray.

Vaginal birth workflow using embodiments of the devices and methods according to the present invention can include removing collection drapes and other pads from beneath the woman and placing the exemplary measurement device under the mother's hips. In a hospital birth, the delivery bed is typically “broken,” and the foot of the bed is removed from the middle section of the bed. Devices according to embodiments of the present invention can be used whether or not the bed is broken. If the bed is broken, the measurement device can rest on the middle section of the hospital bed, with the collection basin extending past the edge of the middle section. If the bed is not broken, the foot of the bed may need to be lowered slightly to accommodate placement of the measurement device. The device can also be used at any point after delivery when accurate measurement of blood loss is needed (e.g., a delayed postpartum hemorrhage).

The device can include a removable insert having a mesh and/or colander filtration structure. The insert can be positioned within the opening leading into the interior volume formed by the device and allows for removal of clots or other large particles that may have traditionally resulted in inaccuracies in measurement the amount of blood loss of the patient. The insert can include a handle that fits over the edge of the device to ensure the position of the insert is maintained during use. The user can remove the insert (and the clots/large particles caught by the mesh) to dispose of the filtered particles prior to visually determining the amount of blood collected in the interior volume of the device. In some embodiments, clots can be removed and weighed/measured separately to be included as part of the blood loss of the patient.

In accordance with embodiments of the present disclosure, an exemplary fluid collection device (e.g., postpartum blood loss collection and measurement device) is provided. The device includes a tray defining a proximal edge and a distal edge, and a basin connected to the proximal edge of the tray. The basin includes an opening at a top perimeter edge and walls extending away from the tray towards a bottom. The walls and bottom define an interior volume configured to receive fluid.

In some embodiments, the tray and basin can be integrally formed or fabricated as a single component. In some embodiments, the tray and basin can be formed or fabricated as independent components. In such embodiments, the basin can be removably connected to the proximal edge of the tray.

The tray is configured to be at least partially positioned over a patient bed or beneath a patient. The tray and the basin can be fabricated from a flexible, rubber material. In some embodiments, the tray can define a substantially planar configuration including flat top and bottom surfaces extending between the proximal and distal edges. In some embodiments, the tray can include opposing side edges extending between the proximal and distal edges, and at least a portion of the side edges tapering inwardly towards the proximal edge. In some embodiments, a width of the proximal edge of the tray can be equal (or substantially equal) to a width of the basin.

In some embodiments, the tray can include fluid guides extending from a top surface of the tray at opposing side edges of the tray. The fluid guides can extend substantially perpendicularly from the top surface of the tray and connect to the opening of the basin. In some embodiments, the tray can include fluid guides extending from a top surface of the tray in a central region of the tray between opposing side edges of the tray. At least one of the walls of the basin can include gradations formed therein or marked thereon to indicate a level of fluid within the interior volume of the basin. In some embodiments, at least three of the walls of the basin can taper inwardly towards the bottom. In some embodiments, a drain line can extend from the bottom of the basin for selective drainage of the fluid from the basin. In some embodiments, a valve can be incorporated into the drain line for selectively draining the fluid from the basin.

In accordance with embodiments of the present disclosure, an exemplary fluid collection system is provided. The system can include a fluid collection device and an insert. The fluid collection device can include a tray defining a proximal edge and a distal edge, and a basin connected to the proximal edge of the tray. The basin can include an opening at a top perimeter edge and walls extending away from the tray towards a bottom, the walls defining an interior volume configured to receive fluid. The insert is configured to be at least partially removably positioned within at least one of the opening or the interior volume of the basin. The insert includes a filtration structure for filtering large particles from the fluid collected in the interior volume of the basin.

The insert can include a handle capable of being releasably engaged with a perimeter lip surrounding the basin. The fluid collection device can include a drain line extending from the bottom of the basin for selective drainage of the fluid from the basin.

In accordance with embodiments of the present disclosure, an exemplary method of fluid collection is provided. The method includes positioning a fluid collection device at least partially on a bed or beneath a patient. The fluid collection device includes a tray defining a proximal edge and a distal edge, and a basin connected to the proximal edge of the tray. The basin includes an opening at a top perimeter edge and walls extending away from the tray towards a bottom, the walls defining an interior volume. The method includes allowing fluid to drain along the tray towards the basin. The method includes collecting the fluid within the interior volume of the basin.

The method can include positioning an insert including a filtration structure within at least one of the opening or the interior volume of the basin, capturing large particles from the fluid with the filtration structure, and removing the insert from the opening or the interior volume of the basin to dispose of the captured large particles. The method includes visually measuring a level of the fluid collected within the interior volume of the basin using gradations formed in or marked on at least one of the walls of the basin.

Any combination and/or permutation of embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the postpartum collection and measurement device, reference is made to the accompanying figures. While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

FIG. 1 is a perspective view of an exemplary postpartum collection and measurement device according to embodiments of the present disclosure;

FIG. 2 is a perspective view of an exemplary postpartum collection and measurement device of FIG. 1;

FIG. 3 is a top view of an exemplary postpartum collection and measurement device of FIG. 1;

FIG. 4 is a side view of an exemplary postpartum collection and measurement device of FIG. 1;

FIG. 5 is a perspective view of an exemplary postpartum collection and measurement device of FIG. 1;

FIG. 6 is a perspective view of an exemplary postpartum collection and measurement device of FIG. 1;

FIG. 7 is a diagrammatic top view of an exemplary postpartum collection and measurement device of FIG. 1;

FIG. 8 is a diagrammatic perspective view of an exemplary postpartum collection and measurement device of FIG. 1;

FIG. 9 is a flowchart of a method of collecting and measuring blood according with an exemplary postpartum collection measurement device according to embodiments of the present disclosure;

FIG. 10 is a perspective view of an exemplary postpartum collection and measurement device according to embodiments of the present disclosure;

FIG. 11 is a top view of an exemplary postpartum collection and measurement device of FIG. 10;

FIG. 12 is a side view of an exemplary postpartum collection and measurement device of FIG. 10;

FIG. 13 is a perspective view of an exemplary postpartum collection and measurement device of FIG. 10 including an insert;

FIG. 14 is a top view of an exemplary postpartum collection and measurement device of FIG. 10 including an insert;

FIG. 15 is a side view of an exemplary postpartum collection and measurement device of FIG. 10 including an insert;

FIG. 16 is perspective view of an insert for an exemplary postpartum collection and measurement device of FIG. 10;

FIG. 17 is an exploded view of an insert of FIG. 16;

FIG. 18 is a perspective view of an exemplary postpartum collection and measurement device according to embodiments of the present disclosure;

FIG. 19 is a top view of an exemplary postpartum collection and measurement device of FIG. 18;

FIG. 20 is a side view of an exemplary postpartum collection and measurement device of FIG. 18;

FIG. 21 is a perspective view of an exemplary postpartum collection and measurement device according to embodiments of the present disclosure;

FIG. 22 is a top view of an exemplary postpartum collection and measurement device of FIG. 21;

FIG. 23 is a side view of an exemplary postpartum collection and measurement device of FIG. 21;

FIG. 24 is a perspective view of an exemplary postpartum collection and measurement device according to embodiments of the present disclosure;

FIG. 25 is a top view of an exemplary postpartum collection and measurement device of FIG. 24;

FIG. 26 is a side view of an exemplary postpartum collection and measurement device of FIG. 24;

FIG. 27 is a perspective view of an exemplary postpartum collection and measurement device according to embodiments of the present disclosure;

FIG. 28 is a top view of an exemplary postpartum collection and measurement device of FIG. 27; and

FIG. 29 is a side view of an exemplary postpartum collection and measurement device of FIG. 27.

### DETAILED DESCRIPTION

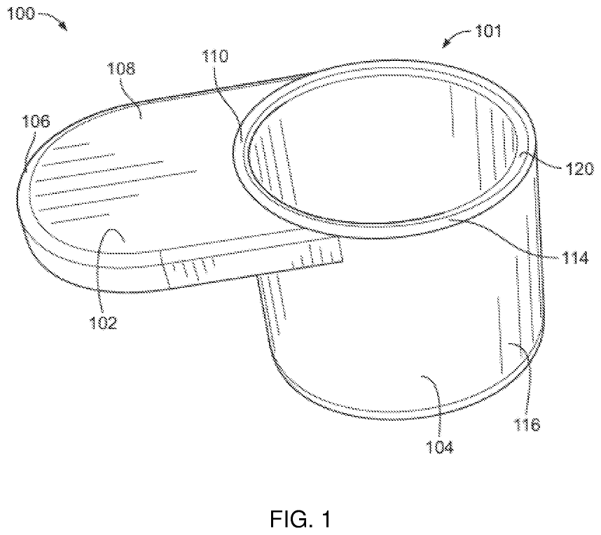

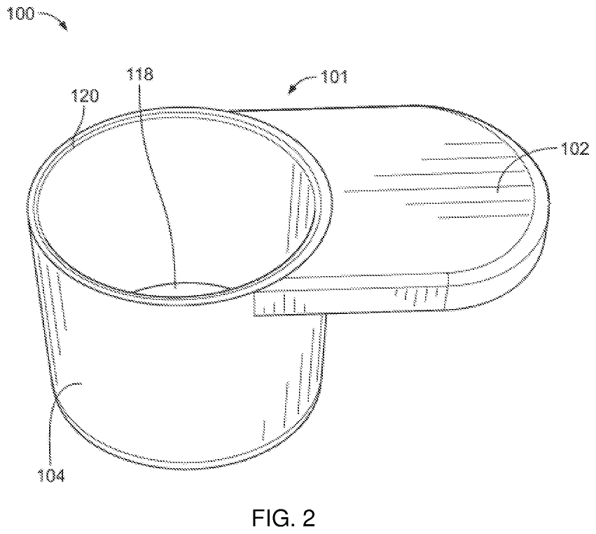

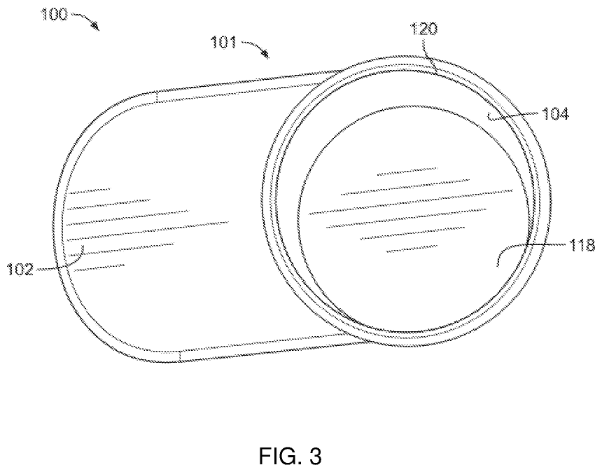

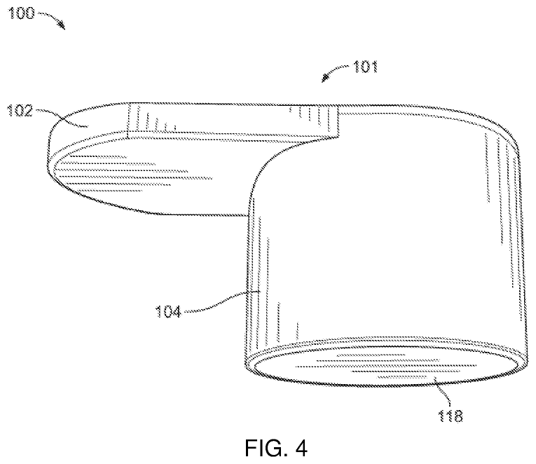

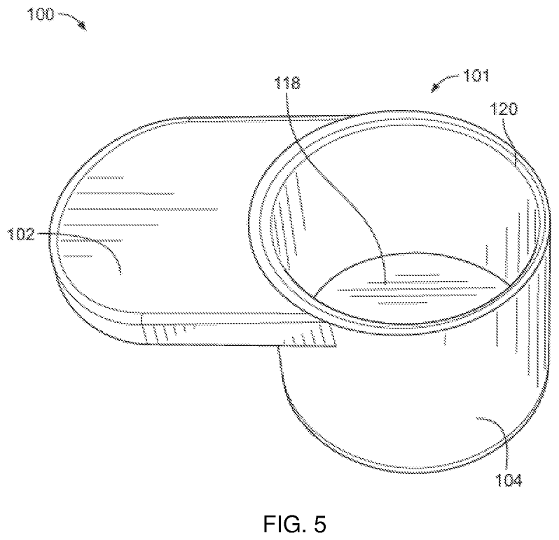

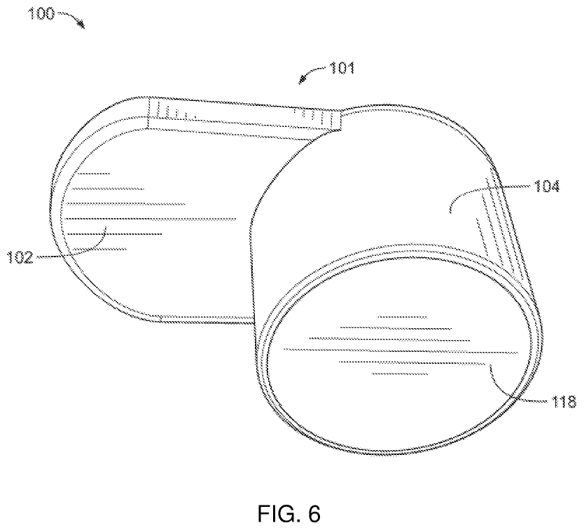

With reference to FIGS. 1-8, perspective, top, side and diagrammatic views of an exemplary blood collection and measurement device 100 (hereinafter “device 100”) are provided. The device 100 includes a body 101 with a tray 102 (e.g., a bill) and a basin 104 (e.g., a cap, a collection section, or the like). The device 100 can be manipulated via tray 102, and fluids can be collected, retained, and/or measured within the basin 104. The tray 102 extends substantially perpendicularly from a top edge of the basin 104, forming a substantially 90° angle therebetween.

The tray 102 can include a grip section 106, a midsection 108, and a lip 110. The lip 110 can define a curved section that connects the tray 102 to the basin 104, thereby defining the proximal end of the tray 102. The grip section 106 can define the opposing distal end of the tray 102. The midsection 108 defines the structure of the tray 102 between the proximal and distal ends. The grip 106 permits manipulation or handling of the device 100, including placement and removal of device 100 relative to the hospital bed and/or patient. The midsection 108 couples or connects the grip 106 with the lip 110.

In some embodiments, the device 100 can be fabricated or formed as a single piece of material such that the tray 102 is integrally formed with the basin 104. For example, the tray 102 and basin 104 can be injection molded or cast together as a single structural component. In some embodiments, the device 100 can be formed as a single structural component using a three-dimensional plastic printer. In some embodiments, the device 100 can be fabricated or formed from two or more pieces of material, with such pieces of material coupled together structurally to ensure stability of the device 100 during use. The device 100 can be fabricated from rubber and/or plastic materials chosen to provide for sufficient stiffness for handling of the device 100 and for collection of fluids in the basin 104, while providing sufficient flexibility for ease of positioning the device at least partially underneath a patient.

As illustrated in FIGS. 1-8, the tray 102 can define an arch-like geometry at the distal end that generally forms the grip section 106, thereby having a substantially semi-circular shape at the distal end edge of substantially rectangular midsection 108. However, it should be understood that the tray 102 may have any geometric configuration that provides a means for securely and conveniently manipulating the device 100. For example, the tray 102 may be shaped to facilitate handling of the device 100 regardless of the amount of fluid within the basin 104, or to ease placement of the device 100 beneath a patient regardless of whether the patient can move.

The grip section 106 can be configured for releasable attachment relative to a hospital bed. In some embodiments, the grip section 106 can be placed at an edge of the bed beneath the patient, with the patient weight maintaining the position of the device 100 relative to the patient and/or the bed without using any additional attachment mechanisms. In some embodiments, the device 100 (or at least the tray 102) can be fabricated from a rubber or silicone material to increase friction against the tray 102, reducing or preventing movement of the tray 102 relative to the patient and/or the bed. In some embodiments, the grip 106 can include one or more securing mechanisms configured to assist in placement, attachment or stabilization of the device 100 relative to the bed and/or patient. In some embodiments, such securing mechanisms can include, e.g., spring-loaded clips, raised edges, combinations thereof, or the like. In some embodiments, the device 100 can be maintained in the desired position by the weight of the patient resting at least partially over the tray 102 and/or grip 106.

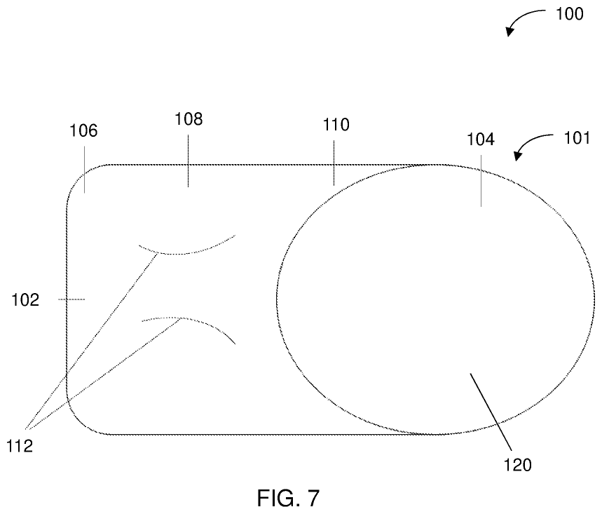

In some embodiments, the tray 102 can include one or more indentations 112 formed in the midsection 108 and extending down the center of the midsection 108 (see, e.g., FIG. 7). In some embodiments, the midsection 108 can include multiple indentations 112. In some embodiments, the midsection 108 can be solid (e.g., no indentations 112). The indentations 112 can be in the form of downwardly formed grooves that create a cavity or passage in the central area of the midsection 108 that is angled towards the basin 104 to encourage flow of fluids towards and into the basin 104, thereby minimizing pooling of the fluid on the tray 102.

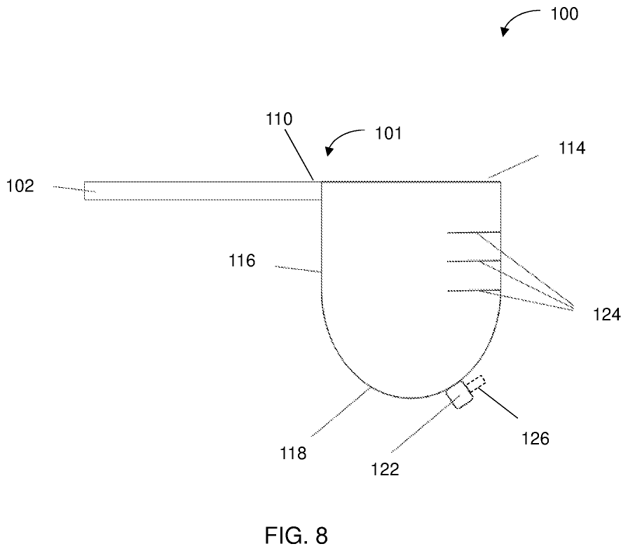

The basin 104 generally includes a top surface or edge 114 that is coupled or connected to the lip 110 of the tray 102 (see, e.g., FIG. 8). The basin 104 includes walls 116 that extend downwardly and connect the top surface or edge 114 to a bottom 118 of the basin 104. In some embodiments, the basin 104 can define a substantially elliptical or cylindrical geometry defined by the curvature of walls 116. However, it should be understood that the basin 104 can have any geometric configuration that provides a means for collecting and measuring fluids. For example, the basin 104 may be shaped to facilitate specific handling of the device 100, or to ease removal of the device 100 from beneath a patient.

As shown by FIGS. 1-8, the top surface or edge 114 includes an opening 120 extending into the body of the basin 104, thereby forming an open cavity into which fluids can flow and pool or collect on the enclosed bottom 118. As fluids flow into the basin 104, the fluid pools on the bottom 118 and gradually rises along the height of the walls 116 towards the top surface or edge 114. In some embodiments, the top surface or edge 114 can include a plug and/or valve capable of being selectively positioned in an open configuration and a closed configuration to allow the basin 104 to be drained without removing the device 100 from under the patient.

In some embodiments, the bottom 118 can include a nozzle 122 extending from the bottom 118. The nozzle 122 can be a tube (e.g., a substantially transparent tube or line) that is fluidly connected to the interior of the basin 104. The nozzle 122 can be selectively actuated into an open (e.g., flow) configuration and a closed (e.g., no flow) configuration that allows the basin 104 to be emptied without removal of the device 100 from the bed and/or patient. In some embodiments, the nozzle 122 can include a valve 126 capable of being selectively actuated for drainage of the basin 104. For example, the nozzle 122 can be fluidly coupled to an exterior drain line for draining the fluid from the basin 104. Such drainage of the basin 104 can be performed when the basin 104 is filled or nearly filled, but bleeding is still ongoing. In some embodiments, the top surface or edge 114 can be compatible with a removable lid (not shown) or other cover for the basin 104, e.g., to control any potential overflow of fluids from the basin 104.

As shown in FIG. 8, the wall 116 of the basin 104 can include a plurality of gradation markings 124 that facilitate visually measuring the amount of fluid collected within the basin 104. The precise number, arrangement, and/or measurements indicated by gradation markings 124 can vary depending on the specificity of the measurements desired. In some embodiments, the device 100 can be designed to contain and measure about 1.000 mL of fluid, with the wall 116 including gradation markings 124 appropriately arranged to indicate at least 0.500 mL, 0.750 mL, and 1.000 mL of fluid. In some embodiments, the device 100 can be designed to contain and measure about 2.500 mL of fluid, with appropriate gradation markings 124 arranged on the wall 116. The basin 104 can collect and retain fluid within the interior cavity or chamber defined by the geometry of the wall 116 between the top surface or edge 114 and the bottom 118. The exact arrangement of the gradation markings 124 may therefore also vary based on the precise geometric configuration of the basin 104. Persons of skill in the art will recognize that the device 100 may be designed to contain and measure any volume of fluid, regardless of the precise geometric configuration of the basin 104.

In some embodiments, the tray 102 can be dimensioned approximately 6 inches long (as measured between the lip 110 and the grip section 106) and approximately 0.25 inches thick (as measured between the top and bottom surfaces of the tray 102). In some embodiments, the grip section 106 can be dimensioned approximately 8 inches wide (as measured between the side edges of the tray 102), and the tray 102 can gradually taper to match the width of the lip 110. In some embodiments, the tray 102 can have alternative shapes or dimensions, such that the tray 102 is larger or smaller. The lip 110 can be configured to smoothly attach the tray 102 and the basin 104. In some embodiments, the edges or corners may be rounded (as shown) or may include sharper connections. In some embodiments, the basin 104 can be approximately 8 inches deep (as measured between the top surface or edge 114 and the bottom 118) and can a diameter of approximately 6 inches (as measured between the walls 116), such that the device 100 defines a total length of approximately 12 inches. Persons of skill in the art will recognize that these dimensions may be adjusted, and that not all embodiments will include the measurements provided by these exemplary embodiments.

In some embodiments, the device 100 can be formed or fabricated from a medical-grade plastic or other non-absorbent material(s) appropriate for medical use. In some embodiments, the device 100 can be formed or fabricated from a medical-grade plastic capable of repeated sterilization and re-use. In some embodiments, the device 100 can be disposable and formed of non-absorbent materials appropriate for one-time medical use. In some embodiments, the device 100 can be formed or fabricated from a combination of materials, or both reusable and disposable portions. For example, in some embodiments, the tray 102 can be formed or fabricated from disposable materials while the basin 104 is formed or fabricated from reusable materials.

The device 100 is configured to permit measuring the amount of fluid collected within the basin 104 without moving the device 100 until the basin 104 is full or substantially full. In some embodiments, the device 100 can formed or fabricated from transparent materials, opaque materials, or a combination of transparent and opaque materials to provide for improved visibility of the contents within the basin 104. For example, if the basin 104 is formed or fabricated from transparent materials, the gradation markings 124 may be visible on the inside of the wall 116 (e.g., similar to a urine “hat” placed in the toilet). In some embodiments, the gradation markings 124 can be on the outside of wall 116, the inside of the wall 116, or both. In some embodiments, if the device 100 is formed or fabricated from non-transparent materials, the wall 116 can be fabricated to define a sufficiently small thickness that would permit visualizing the level of fluid within the basin 104 through the wall 114 from the outside of the device 100. In some embodiments, at least the basin 104 and/or at least the wall 116 can be formed or fabricated from transparent materials, such as plastic, while the remaining sections of the device 100 are formed or fabricated from a non-transparent material.

In some embodiments, the basin 104 can be formed or fabricated from a medical-grade plastic configured to extend below the tray 102 and collect blood from the patient. In some embodiments, the basin 104 can be uniformly molded with the tray 102 or otherwise permanently coupled with the tray 102. In some embodiments, the basin 104 can be releasably attached to the tray 102 (e.g., in embodiments where the basin 104 is designed to be disposable and the tray 102 is designed to be reusable).

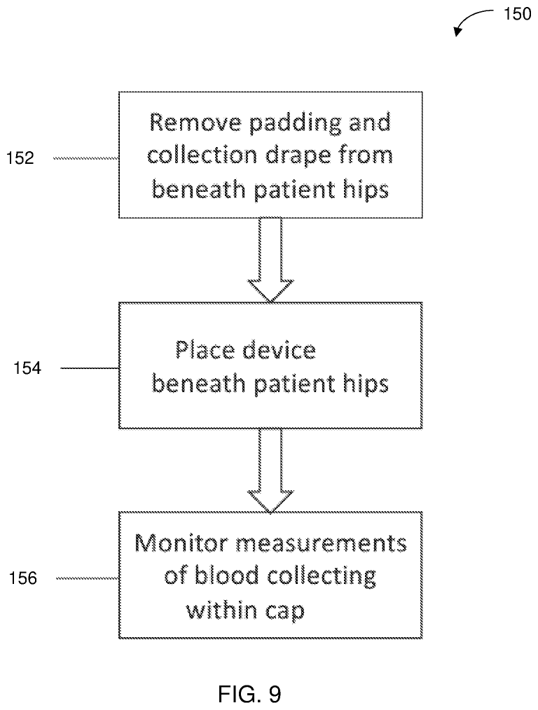

FIG. 9 provides a flowchart depicting an exemplary method 150 of using the devices discussed herein. At step 152, after delivery, the padding and collection drapes can be removed from underneath the patient. At step 154, the device is placed in an operable position, e.g., under a patient's hips. At step 156, medical personnel can monitor the amount of blood collecting within basin of the device and use such measurements to aid in real-time decision making for treatment of the patient.

In some embodiments, the devices discussed herein can be used during delivery (e.g., if a patient is hemorrhaging during or before labor), with the same method described for postpartum use. In such scenarios, the device may need to be removed for the birth of the baby and repositioned once the patient is no longer in labor.

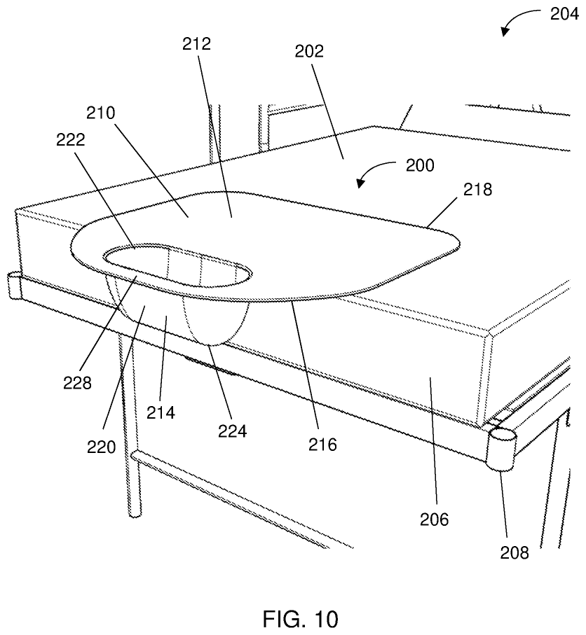

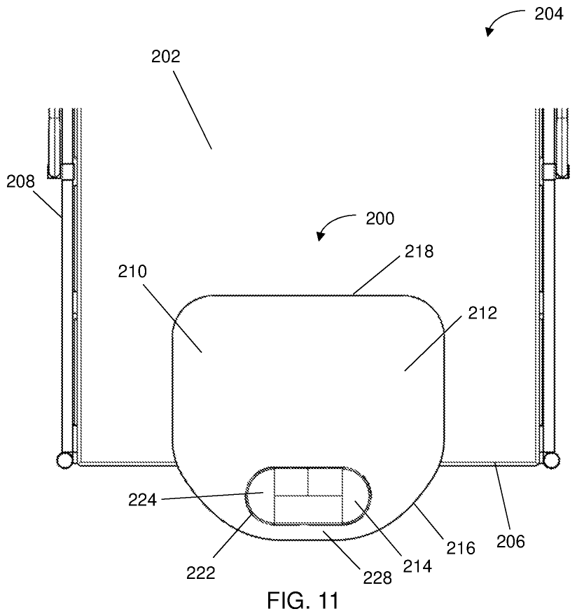

With reference to FIGS. 10-12, perspective, top and side views of an exemplary postpartum collection and measurement device 200 (hereinafter “device 200”) are provided. The device 200 can be substantially similar in structure and/or function to the device 100, except for the distinctions noted herein. It should be understood that one or more features of the devices discussed herein can be substituted and/or combined to achieve a device that effectively and conveniently allows for collection and measurement of blood loss from the patient. The device 200 is configured for positioning on a mattress 202 of a hospital bed 204, the mattress 202 having a front edge 206 and the hospital bed 204 including a frame 208.

The device 200 includes a body 210 with a tray 212 and a basin 214. The tray 212 can define a substantially planar/flat, sheet-like form that extends along a single horizontal plane between proximal and distal edges 216, 218. In some embodiments, the proximal edge 216 can be curved towards the basin 214, providing space around the device 200 for medical professionals treating the patient. In some embodiments, the distal edge 218 can define a substantially linear edge. In some embodiments, the length of the tray 212 as measured between the basin 214 and the distal edge 218 can be, e.g., about 18 inches. In some embodiments, the width of the tray 212 as measured between side edges of the tray 212 can be about, e.g., 18 inches. The planar configuration of the tray 212 allows for positioning of the tray 212 on the top surface of the mattress 202 and at least partially underneath the patient, with the patient weight and/or friction maintaining the position of the device 200 relative to the mattress 202.

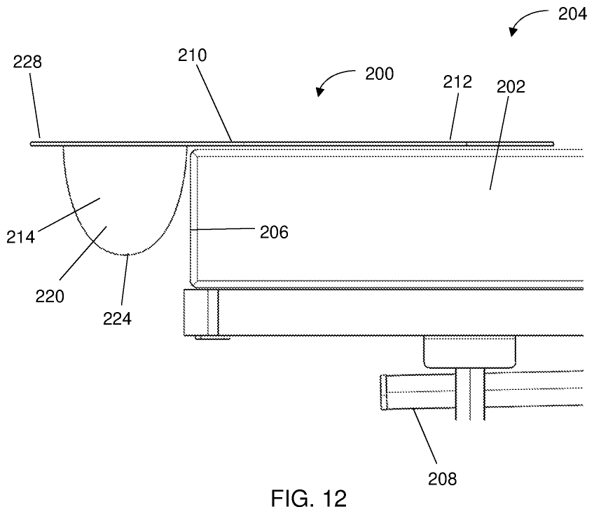

In the embodiment of FIGS. 10-12, the top edge of the basin 214 is substantially aligned with the top surface of the tray 212. The walls 220 of the basin 214 can extend down from the bottom surface of the tray 212 in a substantially perpendicular orientation. The downward and substantially perpendicular extension of the basin 214 from the tray 212 allows for the wall 220 of the basin 214 to be at least partially positioned against the front edge 206 of the mattress 202, with the mattress 202 providing stability to positioning of the device 200 during use with the patient (see, e.g., FIG. 12).

The basin 214 includes an opening 222 at or near the top surface of the tray 212. In some embodiments, the opening 222 can be substantially oval in shape. The opening 222 extends into the interior or cavity formed by the walls 220 of the basin 214, the interior or cavity capable of receiving a predetermined amount of fluid. The configuration of the basin 214 ensures that fluid flowing along the top surface of the tray 212 is guided into the basin 214 through the opening 222. The fluid flows downward to the bottom 224 of the basin 214, allowing the basin 214 to gradually fill with the fluid until the top edge of the basin 214 is reached. The walls 220 of the basin 214 can include graduations formed and/or marked thereon to indicate the level of fluid collected in the basin 214. The width and length of the basin 214 (and the opening 222) are dimensioned smaller than the width and length of the tray 212. In some embodiments, the device 200 can include a lid for covering the opening 222 of the basin 214, thereby preventing or reducing potential spillage of the fluid contained in the basin 214. In some embodiments, the basin 214 can include a drain nozzle extending from the bottom 224 to allow for selective draining of the basin 214 without removal of the device 200 from the hospital bed 204.

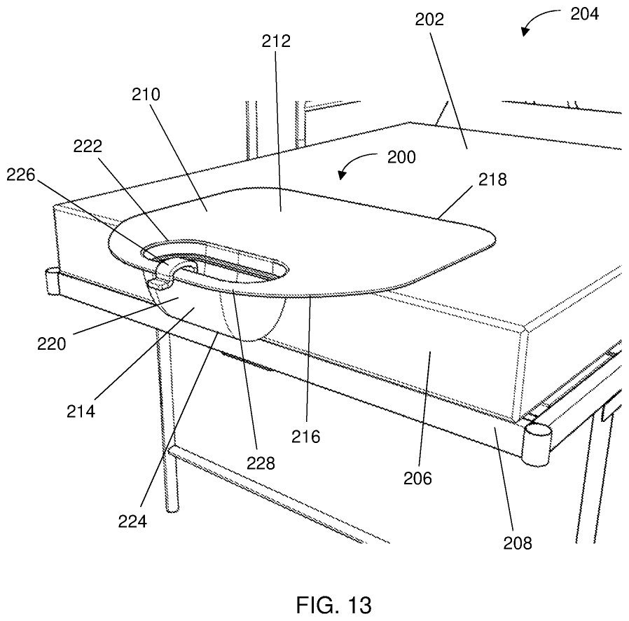

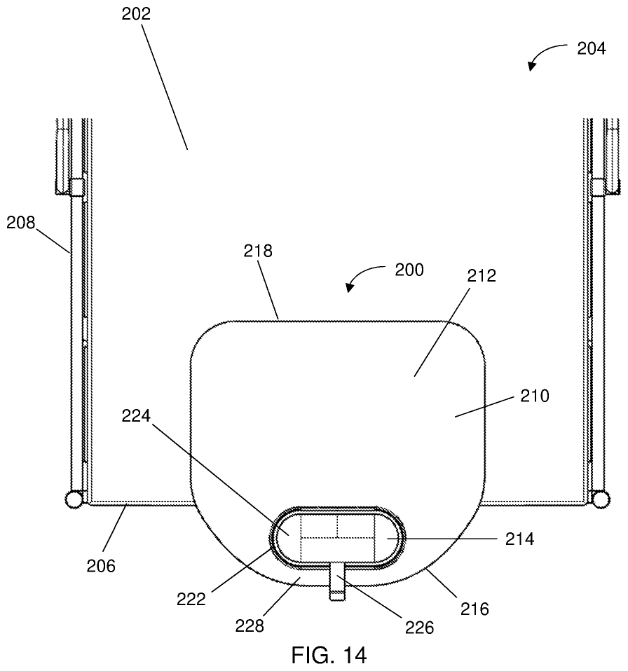

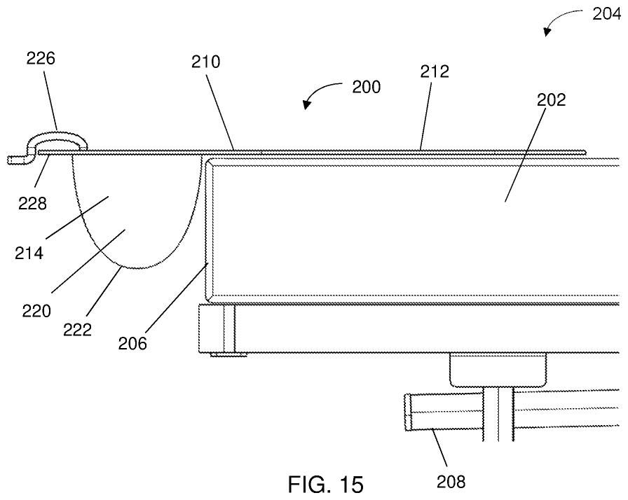

With reference to FIGS. 13-15, in some embodiments, the device 200 can be used in combination with an insert 226. In some embodiments, the insert 226 can assist in capturing large particles, such as gauze, clots, feces, or the like, to prevent such particles from falling into the basin 214. As such, only fluid and small particles are capable of flowing into the basin 214, providing a more accurate measurement of the level of fluid within the basin 214 and, in turn, the amount of blood loss of the patient. In some embodiments, the insert 226 can be used to capture blood clots which can be removed and measured/weighed (and taken into account with the level of blood from the basin 214) to more accurately determine the level of blood loss of the patient. The device 200 can include a front edge or lip 228 that can be used for detachably securing the insert 226 relative to the device 200, thereby preventing the insert 226 from falling into the basin 214.

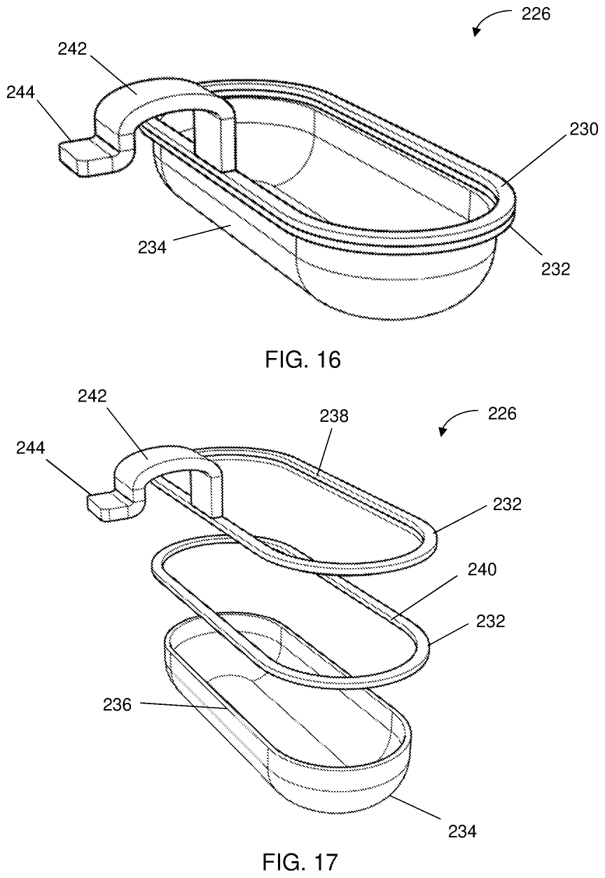

FIGS. 16 and 17 are perspective and exploded views of the insert 226. The insert 226 includes a top frame 230, a bottom frame 232 and a filtration structure 234. The top and bottom frames 230, 232 are configured to receive and secure a top, perimeter edge 236 of the filtration structure 234 in-between the frames 230, 232. The top frame 230 includes a central opening 238 and the bottom frame 232 includes a central opening 240 which allow passage of the filtration structure 234 therethrough. The filtration structure 234 can be, e.g., a plastic mesh, a metal mesh, a flexible mesh, a rigid mesh, combinations thereof, or the like. Although illustrated as defining a trough-shaped configuration, the filtration structure 234 can be a substantially flat, stretched structure covering the openings 238, 240. The top frame 230 includes a handle 242 extending therefrom. The handle 242 can include a hook-like extension 244 at a distal end of the handle 242. The handle 242 can be secured around the edge or lip 228 of the device 200 to maintain the position of the insert 226 in the basin 214.

The device 200 can therefore be used to collect blood and other fluid(s) in the basin 214 during or post labor. The insert 226 can be used to filter out large particles that traditionally result in inaccurate measurement of blood loss. As such, only blood and other fluid(s) enter the basin 214 and the insert 226 can be removed to dispose of the larger particles prior to being replaced on the device 200. The graduations on the basin 214 can be used throughout the procedure to visually measure the amount of blood collected in the basin 214 in real-time. The drain valve or line at the bottom 218 of the basin 214 can be intermittently used to drain the basin 214. Alternatively, the device 200 can be removed from the bed 204 for draining of the basin 214, and subsequently replaced under the patient. In some embodiments, the basin 214 can be removable from the tray 212, allowing for removal of the basin 214 without removal of the tray 212. The device 200 therefore provides a convenient, accurate and cost-effective means for measuring blood loss of the patient.

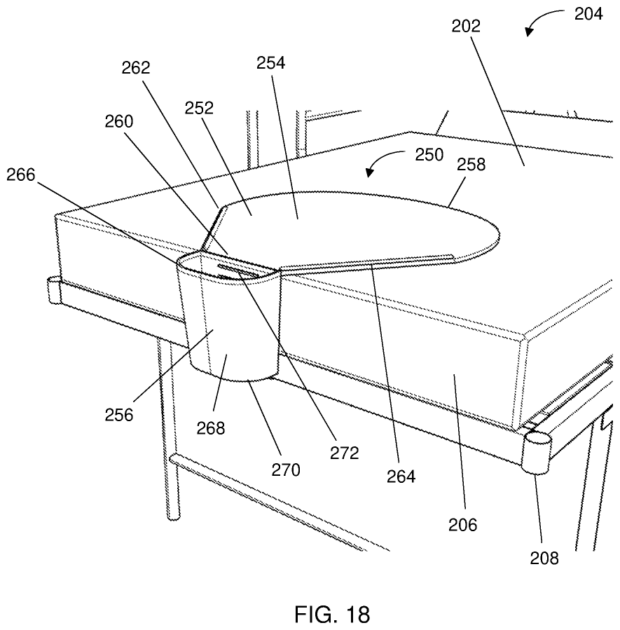

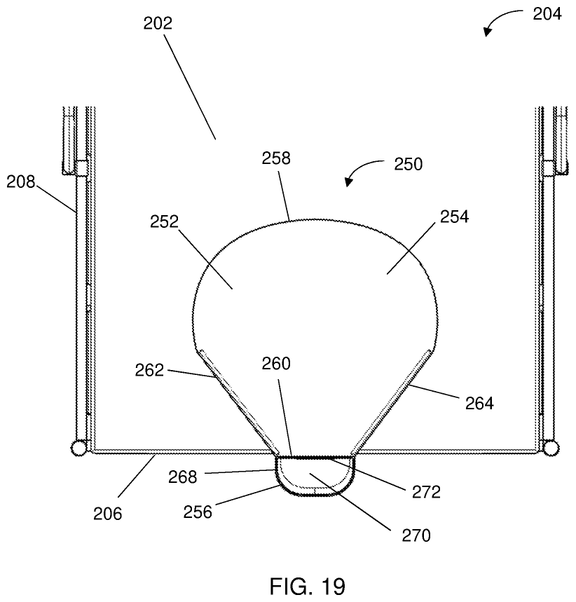

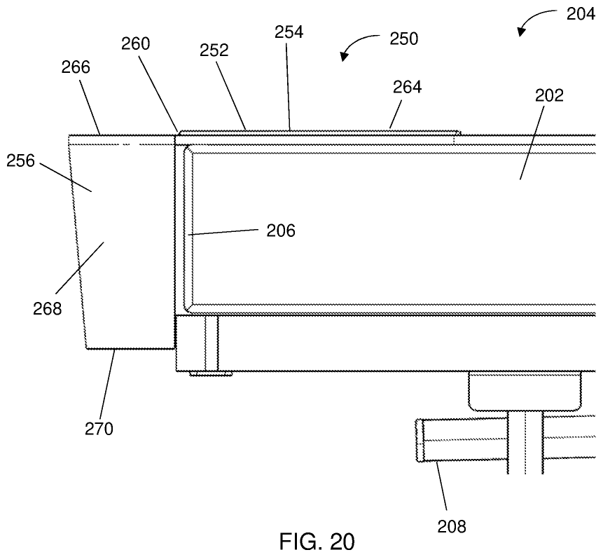

With reference to FIGS. 18-21, perspective, top and side views of an exemplary postpartum collection and measurement device 250 (hereinafter “device 250”) are provided. The device 250 can be substantially similar in structure and/or function to the devices 100, 200, except for the distinctions discussed herein. The device 250 includes a body 252 with a tray 254 and a basin 256. Rather than a substantially linear distal edge of the tray 254, the distal edge 258 can be semi-circular or rounded. The side edges of the tray 254 can taper gradually inward in a linear manner to match the width of the basin 256 at the proximal edge 260 of the tray 254. The tray 254 can include fluid guides 262, 264 (e.g., raised walls, raised edges, raised lips, raised tunnels, or the like) extending along the side edges of the tray 254 that assist with guiding the fluid towards the basin 256. The fluid guides 262, 264 can prevent or reduce spillage of the fluid over the edges of the tray 254.

The basin 256 includes an opening 266 having a substantially linear side at the proximal edge 254, and a semi-circular or rounded opposing side that connects on either side of the proximal edge 254. The basin 256 includes walls 268 extending from the top edge formed by the opening 266 to a bottom 270 surface, defining a volume that can receive therein fluid(s) from the patient. In some embodiments, the tray 154 can be about 18 inches wide at or near the distal edge 258, and about 6 inches wide at or near the proximal edge 260. The basin 256 can similarly be about 6 inches wide, about 3 inches long, and about 6 inches deep. The wide tray 154 at the distal edge 258 allows for positioning of the device 250 under the patient, while the tapered proximal edge 260 results in a narrow basin 256 that provides sufficient volume to capture fluid without interfering with staff tasks in the surrounding area. In some embodiments, the basin 256 can include gradations 272 on the inner wall 268 with the outer wall 268 facing away from the mattress 202 being transparent, allowing for visualization of the gradations 272 through the wall 268.

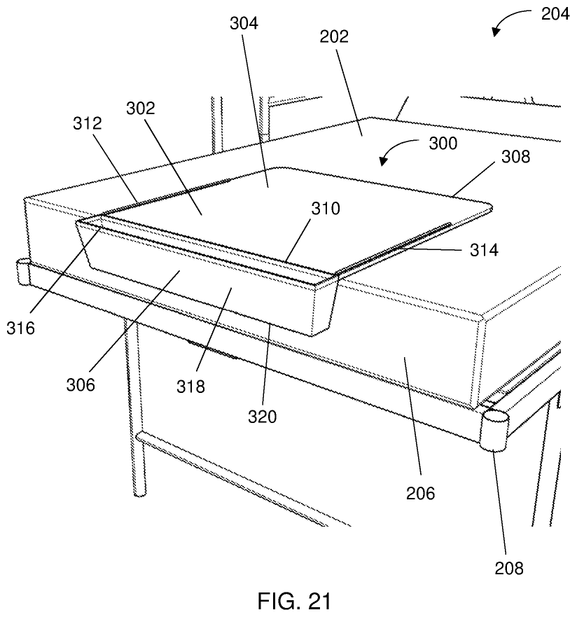

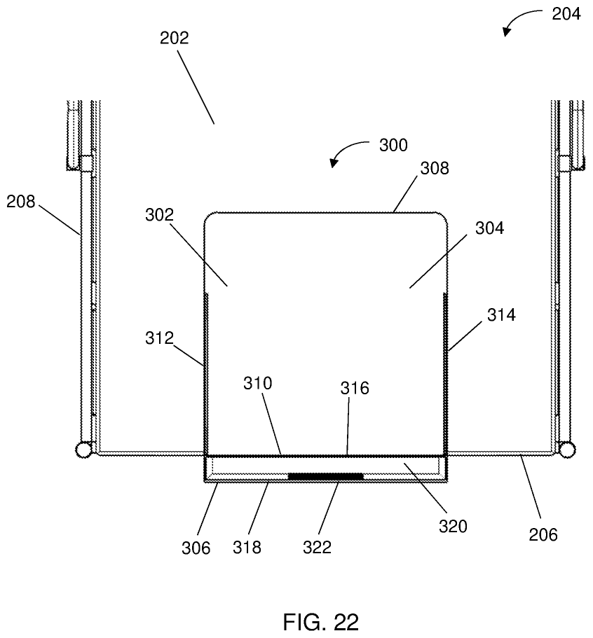

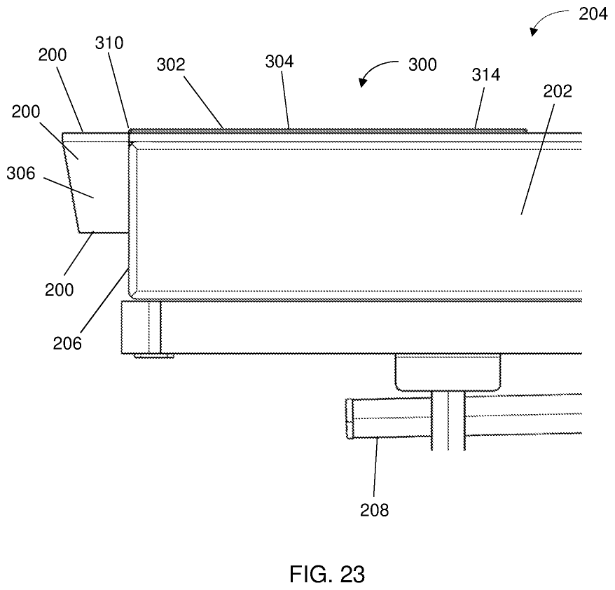

With reference to FIGS. 21-23, perspective, top and side views of an exemplary postpartum collection and measurement device 300 (hereinafter “device 300”) are provided. The device 300 can be substantially similar in structure and/or function to the devices 100, 200, 250, except for the distinctions discussed herein. The device 300 includes a body 302 with a tray 304 and a basin 306. The tray 304 can define a substantially rectangular or square configuration with substantially linear distal and proximal edges 308, 310. In some embodiments, the tray 304 can be about 18 inches long (as measured between the distal and proximal edges 308, 310), and about 18 inches wide (as measured between the side edges). Each of the side edges of the tray 304 can include fluid guides 312, 314 extending substantially perpendicularly from the top surface of the tray 304. The fluid guides 312, 314 can extend at least a portion of the distance from the proximal edge 310 towards the distal edge 308, and assist with guiding fluid towards the basin 306.

The basin 306 includes an opening 316 leading into the interior volume of the basin 306 formed by the walls 318 and bottom 320. The opening 316 can define a substantially rectangular configuration. In some embodiments, the width of the opening 316 can be about 18 inches, the length can be about 2 inches, and the depth can be about 3 inches. The narrow basin 306 prevents interference with staff tasks and the shallow depth of the basin 306 provides more stability when the device 300 is in use. The extended length provides the needed volume to capture fluid from the patient. The basin 306 can include gradations 322 formed or printed on the front surface of the wall 318, providing visibility of the level of fluid in the basin 306.

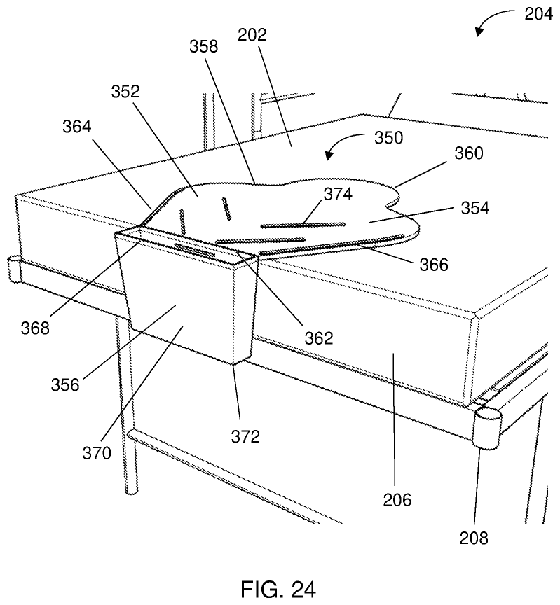

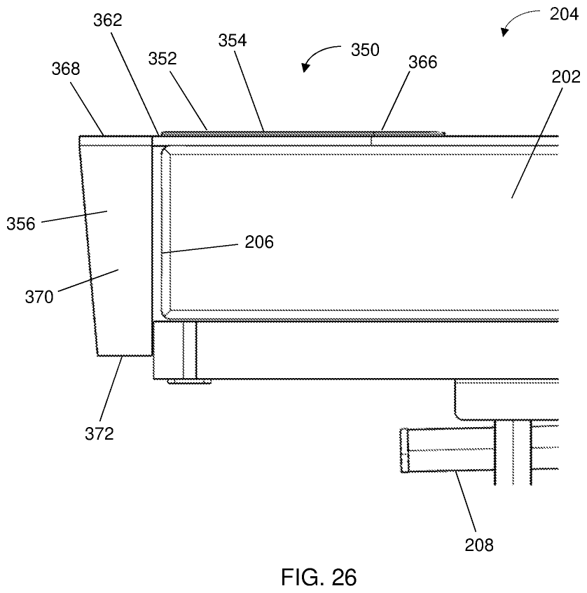

With reference to FIGS. 24-26, perspective, top and side views of an exemplary postpartum collection and measurement device 350 (hereinafter “device 350”) are provided. The device 350 can be substantially similar in structure and/or function to the devices 100, 200, 250, 300, except for the distinctions discussed herein. The device 350 includes a body 352 with a tray 354 and a basin 356. The device 350 includes substantially rounded or curved distal edges 358, and further includes a rounded or semi-circular extension 360 protruding centrally from the distal edge 358. The extension 360 provides additional contour to the tray 354, resulting in greater surface area for maintaining the tray 354 fixed in the desired position beneath the patient. The side edges of the tray 354 can taper gradually towards a linear proximal edge 362 of the tray 354. In some embodiments, the widest area of the tray 354 can be about 18 inches, the length can be about 18 inches, and the width at the proximal edge 362 can be about 9 inches. Each of the side edges includes fluid guides 364, 366 for guiding fluid towards the basin 356.

The basin 356 includes an opening 368 leading into the interior volume of the basin 356 formed by the walls 370 and bottom 372. The opening 368 can define a substantially rectangular configuration. In some embodiments, the width of the opening 368 can be about 9 inches, the length can be about 2 inches, and the depth can be about 6 inches. The narrow basin 356 prevents interference with staff tasks and the shallow depth of the basin 356 provides more stability when the device 350 is in use.

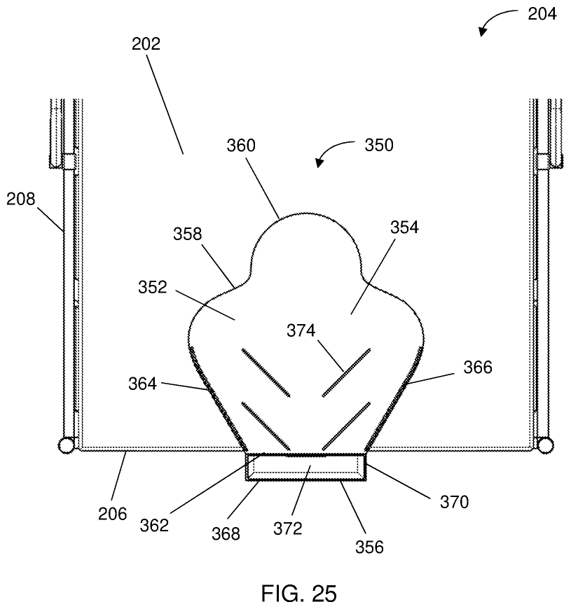

In some embodiments, the tray 354 can include fluid guides 374 (e.g., tapered tray funnels, or the like) extending from the top surface of the tray 354 between the side edges. For example, as illustrated in FIGS. 24 and 25, the tray 354 can include two or more fluid guides 374 oriented at angles relative to each other on opposing sides of a central axis, with the fluid guides 374 guiding fluid towards the central axis and towards the basin 356. Such fluid guides 374 provide more efficient fluid flow into the basin 356 for collection.

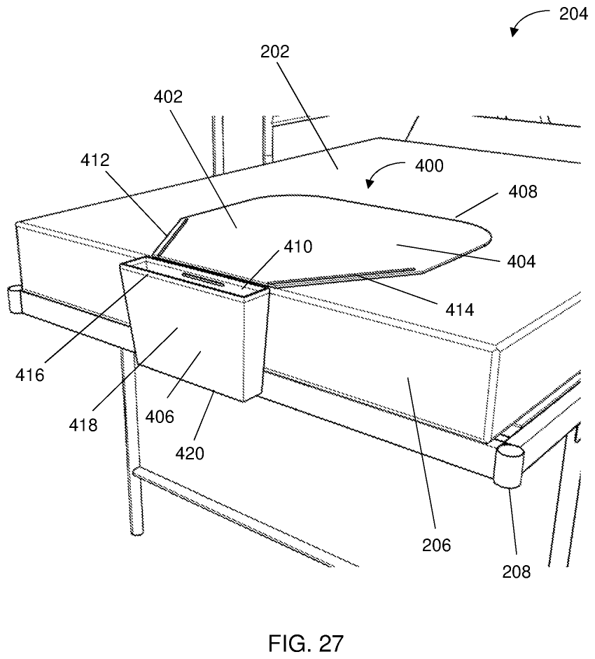

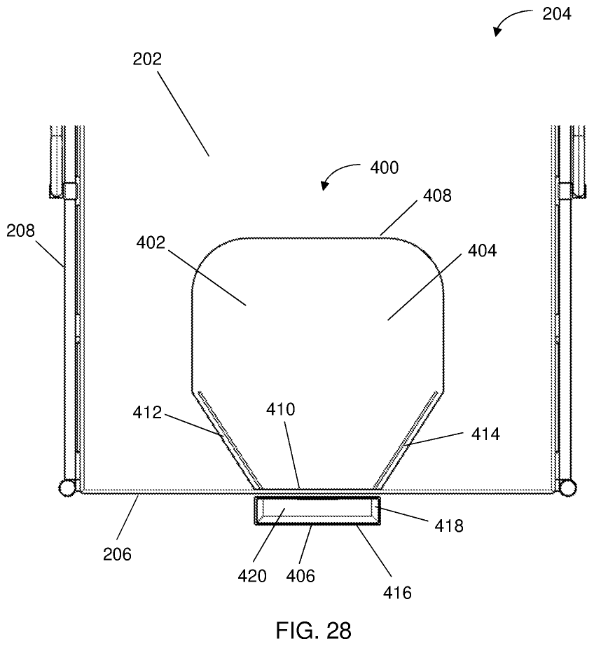

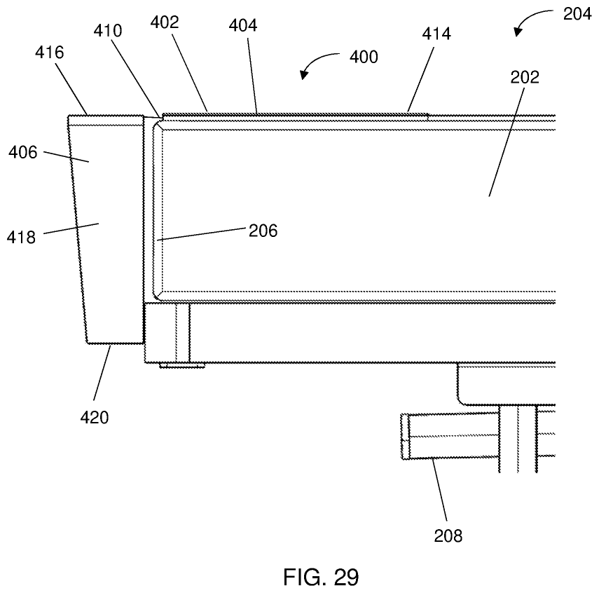

With reference to FIGS. 27-29, perspective, top and side views of an exemplary postpartum collection and measurement device 400 (hereinafter “device 400”) are provided. The device 400 can be substantially similar in structure and/or function to the devices 100, 200, 250, 300, 400, except for the distinctions discussed herein. The device 400 includes a body 402 with a tray 404 and a basin 406. The tray 404 can define a substantially rectangular distal section extending from a linear distal edge 408, and a proximal section extending from the proximal edge 410 with tapered side edges. Each of the side edges can include fluid guides 412, 414 for guiding fluid towards the basin 406. In some embodiments, the width of the tray 404 can be about 18 inches, the length can be about 18 inches, and the width at the proximal edge 410 can be about 9 inches.

The basin 406 includes an opening 416 leading into the interior volume of the basin 406 formed by the walls 418 and bottom 420. The opening 416 can define a substantially rectangular configuration. In some embodiments, the width of the opening 416 can be about 9 inches, the length can be about 2 inches, and the depth can be about 6 inches. The narrow basin 406 prevents interference with staff tasks and the shallow depth of the basin 406 provides more stability when the device 400 is in use. In some embodiments, the rear wall 418 can remain perpendicular relative to the upper perimeter edge of the basin 406, while the front and side walls 418 of the basin 406 can gradually taper towards the bottom 420. In some embodiments, the top perimeter edge surrounding the opening 416 can include fluid guides or a continuous raised lip extending or connecting with the fluid guides 412, 414 to prevent spillage of the fluid over the edge of the basin 406.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A fluid collection device, comprising:
a tray defining a proximal edge and a distal edge; and
a basin connected to the proximal edge of the tray, the basin including an opening at a top perimeter edge and walls extending away from the tray towards a bottom, the walls and bottom defining an interior volume configured to receive fluid. The fluid collection device of claim 1, wherein the tray and basin are integrally formed as a single component.

2. The fluid collection device of claim 1, wherein the tray and basin are formed as independent components, and the basin is removably connected to the proximal edge of the tray.

3. The fluid collection device of claim 1, wherein the tray is configured to be at least partially positioned over a patient bed or beneath a patient.

4. The fluid collection device of claim 1, wherein the tray and the basin are fabricated from a flexible, rubber material.

5. The fluid collection device of claim 1, wherein the tray defines a substantially planar configuration including flat top and bottom surfaces extending between the proximal and distal edges.

6. The fluid collection device of claim 1, wherein the tray includes opposing side edges extending between the proximal and distal edges, and wherein at least a portion of the side edges tapers inwardly towards the proximal edge.

7. The fluid collection device of claim 1, wherein a width of the proximal edge of the tray is equal to a width of the basin.

8. The fluid collection device of claim 1, wherein the tray includes fluid guides extending from a top surface of the tray at opposing side edges of the tray.

9. The fluid collection device of claim 8, wherein the fluid guides extend substantially perpendicularly from the top surface of the tray and connect to the opening of the basin.

10. The fluid collection device of claim 1, wherein the tray includes fluid guides extending from a top surface of the tray in a central region of the tray between opposing side edges of the tray.

11. The fluid collection device of claim 1, wherein at least one of the walls of the basin includes gradations formed therein or marked thereon to indicate a level of fluid within the interior volume of the basin.

12. The fluid collection device of claim 1, wherein at least three of the walls of the basin taper inwardly towards the bottom.

13. The fluid collection device of claim 1, comprising a drain line extending from the bottom of the basin for selective drainage of the fluid from the basin.

14. The fluid collection device of claim 13, comprising a valve incorporated into the drain line for selectively draining the fluid from the basin.

15. A fluid collection system, comprising:
a fluid collection device including (i) a tray defining a proximal edge and a distal edge, and (ii) a basin connected to the proximal edge of the tray, the basin including an opening at a top perimeter edge and walls extending away from the tray towards a bottom, the walls defining an interior volume configured to receive fluid; and
an insert configured to be at least partially removably positioned within at least one of the opening or the interior volume of the basin, the insert including a filtration structure for filtering large particles from the fluid collected in the interior volume of the basin.

16. The fluid collection system of claim 15, wherein the insert includes a handle capable of being releasably engaged with a perimeter lip surrounding the basin.

17. The fluid collection system of claim 15, comprising a drain line extending from the bottom of the basin for selective drainage of the fluid from the basin.

18. A method of fluid collection, comprising:
positioning a fluid collection device at least partially on a bed or beneath a patient, the fluid collection device including (i) a tray defining a proximal edge and a distal edge, and (ii) a basin connected to the proximal edge of the tray, the basin including an opening at a top perimeter edge and walls extending away from the tray towards a bottom, the walls defining an interior volume;
allowing fluid to drain along the tray towards the basin; and
collecting the fluid within the interior volume of the basin.

19. The method of claim 18, comprising positioning an insert including a filtration structure within at least one of the opening or the interior volume of the basin, capturing large particles from the fluid with the filtration structure, and removing the insert from the opening or the interior volume of the basin to dispose of the captured large particles.

20. The method of claim 18, comprising visually measuring a level of the fluid collected within the interior volume of the basin using gradations formed in or marked on at least one of the walls of the basin.
